(19) United States
(12) Plant Patent Application Publication
(10) Pub. No.: US 2002/0194654 P1
(43) Pub. Date: Dec. 19, 2002

(54) Raspberry variety named 'Tadmor'

(76) Inventor: Harvey K. Hall, Motueka (NZ)

Correspondence Address: GREENLEE WINNER AND SULLIVAN P C, 5370 MANHATTAN CIRCLE, SUITE 201, BOULDER, CO, 80303, US

(21) Appl. No.: 09/866,444

(22) Filed: May 25, 2001

(51) Int. Cl. A01H 5/00

(52) U.S. Cl. PLT/204

(57) ABSTRACT

A new and distinct floricane fruiting variety of red raspberry (Rubus idaeus L.), named ‘Tadmor’ is described. The variety produces high yields of attractive, uniform sized, large, high flavored, bright red berries. The plant exhibits a semi spine-free spreading growth habit of greater vigor than other similar varieties. The fruit are suitable for consumption as high grade fresh berries and are also amenable to processing. In addition, the plant has displayed resistance to Raspberry Bushy Dwarf Virus (RBDV) under New Zealand conditions.

### BACKGROUND TO THE INVENTION

The new variety of red raspberry, Rubus idaeus L, was created in the course of a planned breeding program carried out at HortResearch Nelson, New Zealand. The parents used to make the cross in 1990 were the selections ORUS 576-47 (seed parent) and 86105N4.4 (pollen parent). The selection ORUS 576-47, since named ‘Lewis’ (not patented) originated from the Oregon State University, United States Department of Agriculture, Agriculture Research Service Rubus breeding program. 86105N4.4, since named ‘Waimea’ (not patented), is a RBDV resistant New Zealand selection.

The parentage of the new variety includes the Scottish varieties ‘Glen Prosen’ (not patented) and ‘Glen Moy’ (not patented ), and the United States varieties ‘Meeker’ (not patented) and ‘Willamette’ (not patented). The background of ‘Tadmor’ also includes the species Rubus pileatus and R. occidentalis via the Scottish breeding program, and the species R. strigosus via the Oregon Rubus breeding program.

Seed from these crosses was grown and the original plant of the new variety was selected during the 1993-94 summer (Southern Hemisphere) and was found to exhibit:

(a) a semi spine-free arching growth habit of outstanding vigor,

(b) the ability to form attractive, even sized large red fruit of good flavor in exceptionally high yields on medium-long fruiting laterals, and

(c) resistance Raspberry Bushy Dwarf Virus (RBDV).

The new variety was first asexually propagated in 1994, reproduced by root cuttings. The resulting plants propagated true to type demonstrating that the characteristics of the new variety are stable and are transmitted without change through succeeding generations.

### SUMMARY OF THE INVENTION

The new variety was tested and evaluated during the years 1995 to 2000 at HortResearch Nelson.

When compared to the parent ‘Lewis’, the new variety is found to form larger, similarly firm fruit in higher yields. ‘Tadmor’ is further distinguished from ‘Lewis’ by having more canes that are thicker and longer, and producing fruit which are lighter in color, with increased shininess and requiring greater force to separate the berry from the plug. When compared to the parent ‘Waimea’, the new variety exhibits larger, richer red colored fruit in higher yields, a later picking date (approximately 7 to 8 days later), and a longer picking period. ‘Tadmor’ is further distinguished from ‘Waimea’ by having a few spines on mature canes and many spines on juvenile canes, greater cane vigor and cane number, and by having fruit which are more difficult to separate the berry from the plug. Data collected during the evaluation period comparing fruiting performance of the new variety with standard New Zealand varieties is presented in Table 1.

TABLE 1
Comparison of fruiting performance.

| | Average Yield | Berry Weight |
|---|---|---|
| Variety | (T/ha) | (g) |
| ‘Tadmor’ | 25.3 | 4.6 |
| ‘Marcy’ | 21.1 | 2.8 |
| ‘Skeena’ | 15.8 | 3.3 |
| ‘Chilliwack’ | 15.0 | 2.9 |
| ‘Waimea’ | 22.3 | 3.3 |

The data presented in Table 1. demonstrates the exceptionally high fruit yield potential of the new variety. In particular, the greater individual berry weight of fruit of the new variety. ‘Tadmor’ berries commonly weigh approximately 4.6 g, with this fruit weight achieved consistently throughout the harvest season. Berries of the new variety are suitable for consumption as high-grade fresh fruit and also are well suited for processing. The color of the processed product is a lighter red than that of ‘Marcy’, ‘Meeker’, ‘Skeena’ or ‘Willamette’, standard varieties for processing in New Zealand or the United States.

### BRIEF DESCRIPTION OF THE DRAWINGS

The accompanying photographs show typical specimens of the new variety in color as true as is reasonably possible. The photographs were prepared in December 2000 and depict three year-old plants and plant parts grown outdoors at HortResearch Nelson, New Zealand.

FIG. 1. Illustrates a fruiting plant of the variety showing the large fruit size and high productivity. The fruiting laterals are shown to be medium length and slightly ascending.

FIG. 2. Illustrates typical plants of the variety showing the spreading nature of the canes at full canopy development.

FIG. 3. Illustrates enlarged close-up side and end views of typical fruit of the variety.

FIG. 4. Illustrates a tip of growing foliage of the variety from a primnocane with the leaflets at various stages of development.

FIG. 5. Illustrates fully opened leaflets of the variety from a floricane showing the upper and lower surface of the leaves.

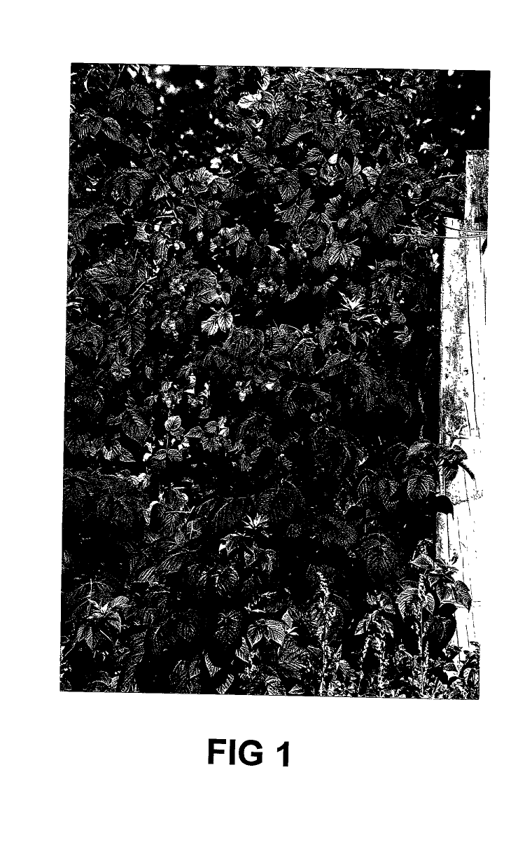

### DETAILED DESCRIPTION

The specimens described were grown at HortResearch Nelson, New Zealand, and the plants were 2 years old in the 2000-2001 season when the observations were made.

Plant and Foliage

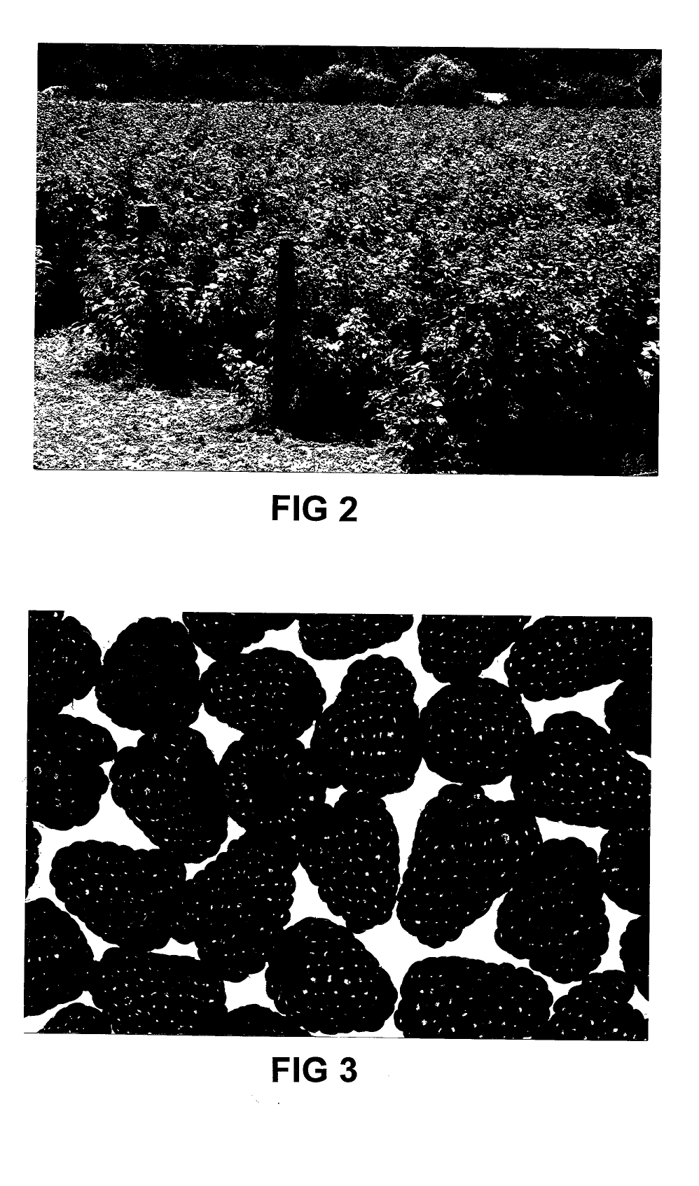

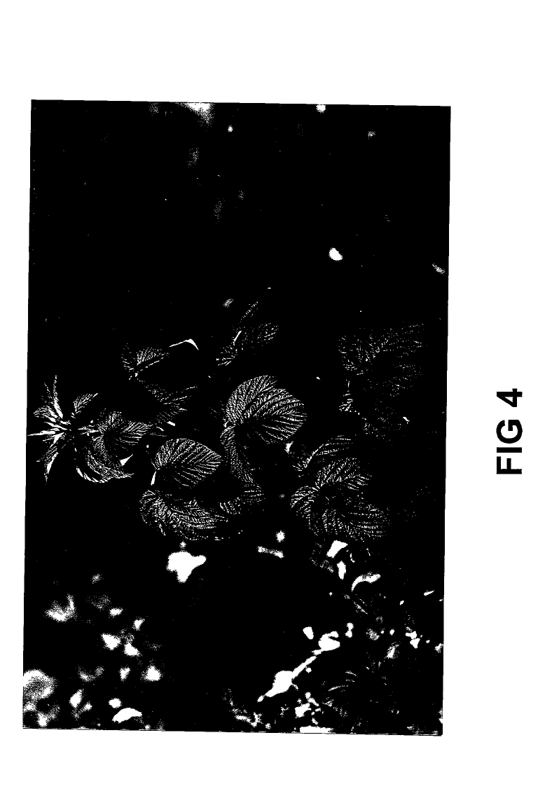

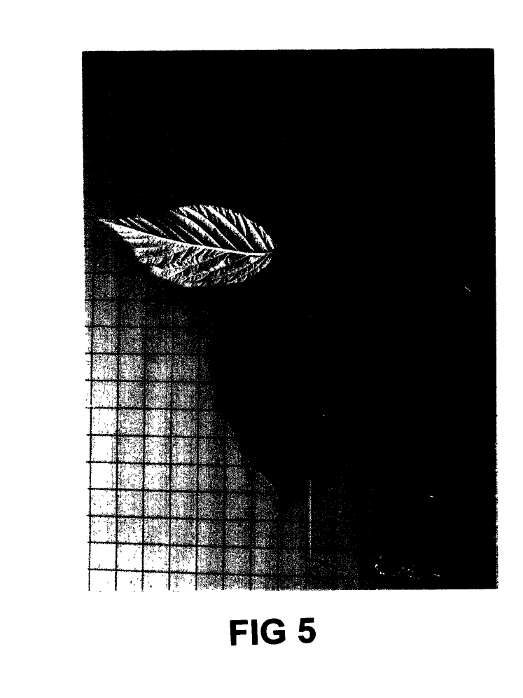

The plant exhibits a spreading growth habit (FIG. 2). Typical mature plants range between 2.0 m to 2.5 m in height, although this may vary with the growing conditions. Very strong vigor is exhibited, manifested as a greater number of canes and longer, thicker canes than similar varieties. Spines are largely absent on mature canes but frequently present on juvenile canes. New shoots commonly possess strong anthocyanin coloration, while mature canes display red coloration in summer and tan coloration during the winter. The canes have a somewhat sparse leaf presentation which tends to provide excellent fruit presentation at harvest time. The leaves are crinkled, almost flat, and moderately glossy (FIGS. 4 and 5). The fruit is borne primarily on the previous year's growth.

Inflorescence

White flowers are borne on short slender pedicels that have few spines. The time of bloom is late-season for a summer-fruiting raspberry. The flowers have five sepals and five petals with the pedicel commonly having a length of approximately 2 to 4 cm. However, the more basal the pedicel the longer it commonly becomes with pedicel lengths up to about 6 cm then being observed. A typical flower diameter is approximately 2.2 cm. The flowers are predominantly borne singly, although sometimes in clusters of two or more. Terminal branch flower clusters frequently consist of two flowers and basal flower clusters may number three to five. The flowers have no discernible fragrance.

Harvest

At HortResearch Nelson, the typical start date for picking the new variety is December 17^{th}. Fifty 50 percent of the harvest is typically completed by January 5 , and harvest ceases approximately February 4^{th}. The date at which 50 percent of harvest is complete is approximately 20 days later for ‘Tadmor’ than for ‘Marcy’. However, the duration of the harvest period for ‘Tadmor’ is similar to ‘Marcy’.

‘Tadmor’ is moderately suitable for harvest by machine due to the moderate ease of removal of the berry from the plug. The fruit sample picked by this method is good quality but it may contain some over-ripe fruit that have not been removed the previous pick. After the passage of the machine there is some fruit retained on the plant even if the plant is shaken more vigorously than is usual with ‘Skeena’, the standard for machine harvest in New Zealand as fruit are harder to remove.

Fruit

The berries formed on ‘Tadmor’ are large in size. Average weight of berries produced at HortResearch Nelson during the 1999-2000 summer was 4.6 g. The fruit is round-conical in configuration (FIG. 3) and is bright red in appearance with a medium to high level of glossiness. The berries are firm and fleshy and of good flavor. The number of drupelets per fruit is commonly approximately 104 and seed weight 1.5 mg.

In shelf life trials ‘Tadmor’ performs better than ‘Marcy’ and ‘Skeena’. ‘Tadmor’ fruit has a better appearance than that of either of these varieties in commercial packs due to the larger, firmer fruit and light, bright color of the berries.

Pest and disease resistance

No data is available on resistance to aphids but ‘Tadmor’ has shown susceptibility to European red mites.

Since the selection of this clone in 1993 numerous tests for raspberry bushy dwarf virus (RBDV) have been done using ELISA but on no occasion has the virus been detected in spite of high infection pressure. From this we suggest that ‘Tadmor’ is likely to be resistant to the common strain of RBDV found in New Zealand.

1. A new and distinct variety of raspberry plant, Rubus idaeus L, substantially as herein shown and described.
